(19) United States
(12) Patent Application Publication
(10) Pub. No.: US 2014/0170699 A1
(43) Pub. Date: Jun. 19, 2014

(54) TEST FOR DETECTING SPOILAGE IN A FLEXIBLE PACKET

(71) Applicant: ABBOTT LABORATORIES, ABBOTT PARK, IL (US)

(72) Inventors: Eric Himmelreich, Westerville, OH (US); Kenneth W. Botterbrodt, Marlton, NJ (US); David C. Ulstad, Dublin, OH (US)

(73) Assignee: ABBOTT LABORATORIES, ABBOTT PARK, IL (US)

(21) Appl. No.: 14/116,252

(22) PCT Filed: May 11, 2012
(86) PCT No.: PCT/US12/37565

§ 371 Date: Feb. 18, 2014

Related U.S. Application Data
(60) Provisional application No. 61/485,368, filed on May 12, 2011.

(51) Int. Cl. G01N 33/02

(52) U.S. Cl. CPC G01N 33/02
USPC 435/34

(57) ABSTRACT

An apparatus for detecting spoilage of the contents of a packet is disclosed. The apparatus includes a pair of holding panels (125, 130) disposed in opposed spaced relationship with each other to define a gap therebetween in which the packet (102) is retained with one packet sidewall facing one holding panel and the other packet sidewall facing the other holding panel. At least one squeezing member (178, 180) is operable to squeeze at least a portion of the packet while the packet is retained between said holding panels. A cutting member (160) is movable to cut at least one inlet in the packet. A probe (150) is moveable relative to the panels for insertion through the at least one inlet in the packet to position the probe within the packet, and the probe is operable to assess at least one characteristic of the contents of the packet indicative of spoilage of the contents of the packet.

### CROSS REFERENCE TO RELATED APPLICATIONS

This application claims priority to U.S. Provisional Application No. 61/485,368, filed May 12, 2011, the disclosure of which is hereby incorporated by reference in its entirety.

### FIELD OF THE DISCLOSURE

The field of the disclosure relates generally to a testing apparatus and methods to determine a characteristic of the contents of a flexible package. More particularly, the testing apparatus may be used to assess one or more characteristics of the contents of the package, for example, pH, density, electrical conductivity, color, moisture content, temperature or the like. Even more particularly, the present disclosure describes such a testing apparatus that is semi-automated and tests for one or more characteristics of the substance while in the package in order to determine whether the contents have spoiled.

### BACKGROUND OF THE DISCLOSURE

Infant nutritionals, adult and medical nutritionals, sport nutritionals, energy gels and the like are often packaged in small flexible pouches, or packets.

As one example, a human milk fortifier may be packaged in a sealed pouch or packet such as those disclosed in U.S. Provisional Patent Application No. 61/427,526, filed Dec. 28, 2010 and PCT Application No. PCT/US2011/064247, filed Dec. 9, 2011, the disclosures of which are incorporated herein by reference in their entireties.

Such nutritional substances are commonly aseptically processed and hermetically sealed to ensure that the contents of the package are not spoiled by foreign substances, such as bacteria, viruses, mold or the like that may otherwise enter the package at the time of sealing. One possibility is the failure of the aseptic process or the hermetic sealing process to have a failure, resulting in the products becoming spoiled. It is also possible for a package to have an incomplete seal, such that the package is not hermetically sealed, thereby allowing the substance contained in the package to become spoiled. Thus, it is important that packages be inspected for spoilation prior to being offered to consumers.

Commonly, pH is used as a measurement for testing attributes of manufactured food products for spoilation. In low acid foods, a change in pH can be an indicator of spoilage. For manufacturers to release their low acid foods with confidence that they are not spoiled, large quantities of destructive pH testing after an incubation period is often required. Such products are typically manufactured in bulk batches, which may include multiple thousands, or millions of units per batch. Typically, the testing process for spoilation is very labor intensive. The manual testing process typically involves multiple steps for each package tested. For example, a worker may have to remove a package from a bulk bin, open the package using a knife, insert a pH testing probe into the package, read the results and record the results by hand. For example, testing for 30,000 packages may take three workers one week of multiple work shifts to complete. As such, the manual process is time consuming, costly and prone to inaccuracies in data reading and recording.

Accordingly, there is an unmet need for a semi-automated pH testing device that increases the safety, throughput, accuracy, and repeatability of such testing.

### SUMMARY OF THE DISCLOSURE

In one aspect, a method of detecting spoilage of the contents of a flexible packet is disclosed. The packet has opposed sidewalls and a longitudinal axis. The method includes locating the packet between a pair of opposed holding panels with the packet oriented generally parallel to the holding panels such that one packet sidewall faces one of said holding panels and the opposite packet sidewall faces the other holding panel. The packet is agitated while retaining the packet between the opposed panels to thereby agitate the contents of the packet. An opening is formed in the packet and a probe is inserted through the opening into the interior of the packet. The probe is operated to assess at least one characteristic of the contents of the packet indicative of spoilage of the contents of the packet.

In another aspect, an apparatus for detecting spoilage of the contents of a flexible packet is disclosed. The packet has opposed sidewalls and a longitudinal axis. The apparatus includes a pair of holding panels disposed in opposed spaced relationship with each other to define a gap therebetween in which the packet is retained with one packet sidewall facing one holding panel and the other packet sidewall facing the other holding panel. At least one squeezing member is operable to squeeze at least a portion of the packet while the packet is retained between said holding panels. A cutting member is movable relative to the holding panels to cut at least one inlet in the packet. A probe is moveable relative to the panels for insertion through the at least one inlet in the packet to position the probe within the packet. The probe is operable to assess at least one characteristic of the contents of the packet indicative of spoilage of the contents of the packet.

In yet another aspect, a testing system for detecting a characteristic of the contents of a plurality of flexible packets is disclosed. The packets each have opposed sidewalls and a longitudinal axis. The system includes a plurality of testing apparatus arranged in close proximity to each other. Each testing apparatus is in communication with a common computer system and capable of transmitting data to the computer system. Each testing apparatus has a testing assembly configured to retain at least one packet therein during testing. A cutting member is movable relative to the holding panels to cut at least one inlet in the packet. A probe is movable relative to the packet for insertion through the at least one inlet in the packet to position the probe within the packet. The probe is operable to assess at least one characteristic of the contents of the packet and communicate the characteristic to the common computer system.

The computer system is operable to control a plurality of testing apparatus. Each testing apparatus includes a cutting member movable relative to the packet to cut at least one inlet in the packet and a probe moveable relative to the packet for insertion through the inlet in the packet to position the probe within the packet. The probe is operable to assess at least one characteristic of the contents of the packet.

### BRIEF DESCRIPTION OF THE DRAWINGS

FIG. 1 is a side elevation of one embodiment of a characteristic testing apparatus.

FIG. 2 is a side elevation of one embodiment of a testing assembly of the characteristic testing apparatus of FIG. 1.

FIG. 3 is a top view of the testing assembly of FIG. 2.

FIG. 4 is a side schematic of the testing assembly of FIG. 2 with a lever arm of the testing assembly in a first angular position.

FIG. 5 is a side schematic thereof with the lever arm in a second angular position and a packet being loaded onto the lever arm.

FIG. 6 is a side schematic thereof with the packet loaded on the lever arm and the lever arm moved back to its first angular position.

FIG. 7 is a side schematic thereof with the lever arm back to the second position to permit unloading of the packet from the lever arm.

FIG. 8 is a side schematic of the testing assembly of FIG. 2 with mixing bars of the assembly in a retracted position.

FIG. 9 is a section of the testing assembly of FIG. 8 taken along the plane of line 9-9.

FIG. 10 is a section of the testing assembly of FIG. 8 taken along the plane of line 10-10.

FIG. 11 is a side schematic of the testing assembly of FIG. 2 with one of the mixing bars in a squeezing configuration.

FIG. 12 is a section of the testing assembly of FIG. 11 taken along the plane of line 12-12.

FIG. 13 is a section of the testing assembly of FIG. 11 taken along the plane of line 13-13.

FIG. 14 is a side schematic of the testing assembly of FIG. 2 with the other one of the mixing bars in a squeezing configuration.

FIG. 15 is a section of the testing assembly of FIG. 14 taken along the plane of line 15-15.

FIG. 16 is a section of the testing assembly of FIG. 14 taken along the plane of line 16-16.

FIG. 17 is a side schematic of the testing assembly of FIG. 2 with a cutting member of the assembly in a retracted position.

FIG. 18 is a front schematic of the testing assembly of FIG. 17.

FIG. 19 is a side schematic of the testing assembly of FIG. 2 with the cutting member in an extended position.

FIG. 20 is a front schematic of the testing assembly of FIG. 19.

FIG. 21 is a side schematic of a probe assembly with a probe thereof in a retracted position.

FIG. 22 is a side schematic illustrating the probe of the probe assembly in a sampling position.

FIG. 23 is a side schematic of the testing assembly of FIG. 2 with a hold down member in a released position.

FIG. 24 is a side schematic of the hold down member in a packet holding configuration.

FIG. 25 is a perspective view of a plurality of testing assemblies.

### DETAILED DESCRIPTION OF THE EMBODIMENTS

For the purposes of promoting an understanding of the principles of the present disclosure, reference will now be made to embodiments and specific language will be used to describe the same. It will nevertheless be understood that no limitation of the scope of the claims is thereby intended, such alteration and further modifications of the readings of the disclosure as illustrated herein being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

Unless otherwise identified, like numerals in the Figures indicate like parts.

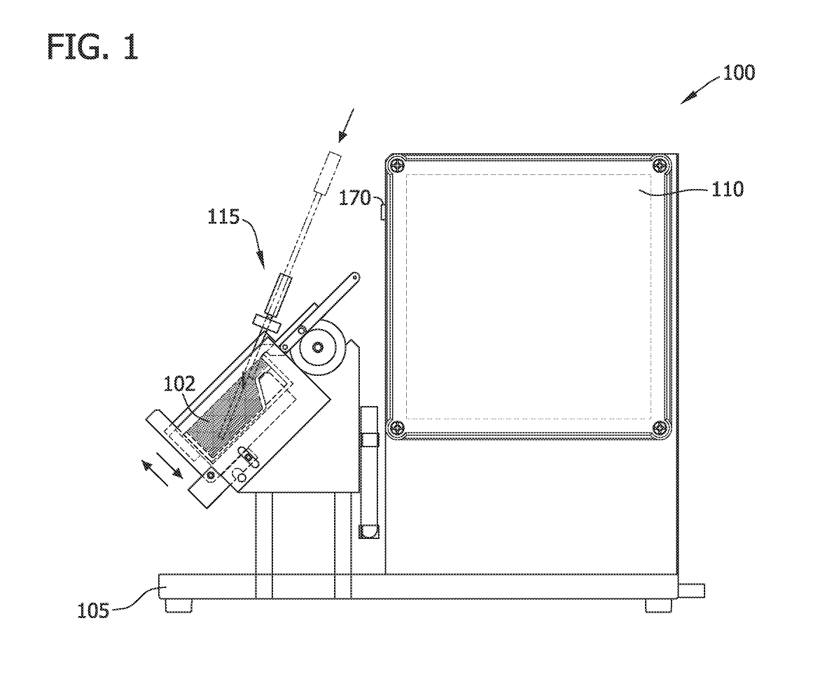

With reference now to the drawings, and in particular to FIG. 1, the testing apparatus for assessing spoilage of a substance is generally indicated at 100. Although the disclosure generally references “spoilage” of the package, characteristics of the substance other than spoilage may be determined as discussed herein. The illustrated apparatus 100 is, in one particularly suitable embodiment, for assessing spoilage of a liquid substance contained in a flexible pouch or packet 102. One such packet is disclosed in U.S. Provisional Patent Application No. 61/427,526, filed Dec. 28, 2010. The testing apparatus 100 generally comprises suitable structure defining a fixed base 105 supporting a control panel housing or box 110 housing a suitable control system (not shown but including, e.g., a programmable logic controller, computer processor, non-transitory memory storing a program thereon, one or more input and/or output devices, circuitry, switches, etc.) for controlling operation of the testing apparatus 100, and a testing assembly 115. In one embodiment, the control system may be capable of being programmed by an operator. In one suitable embodiment, the base 105 is pan shaped and functions as a catch pan for catching liquid from the tested packets 102 or other components of the testing assembly.

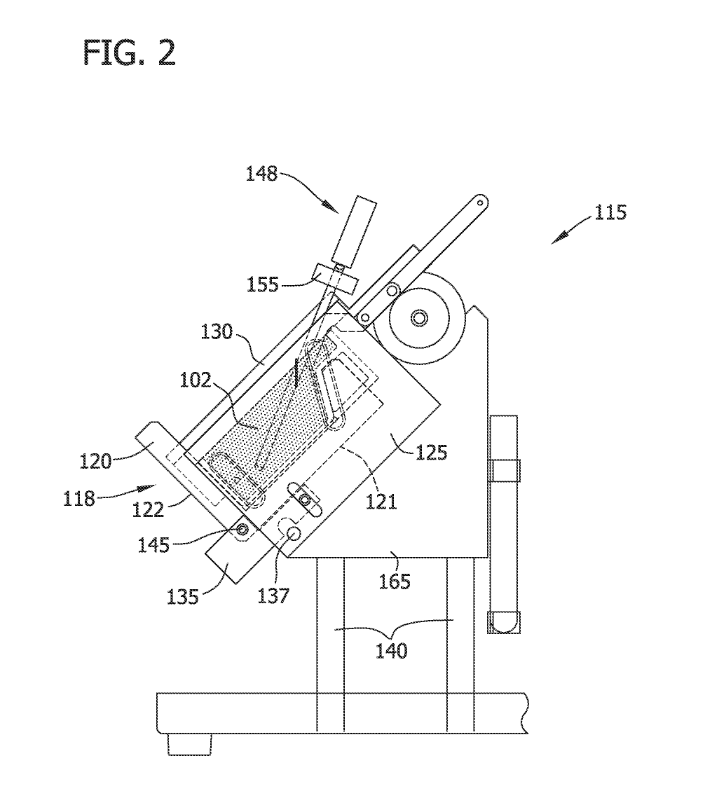

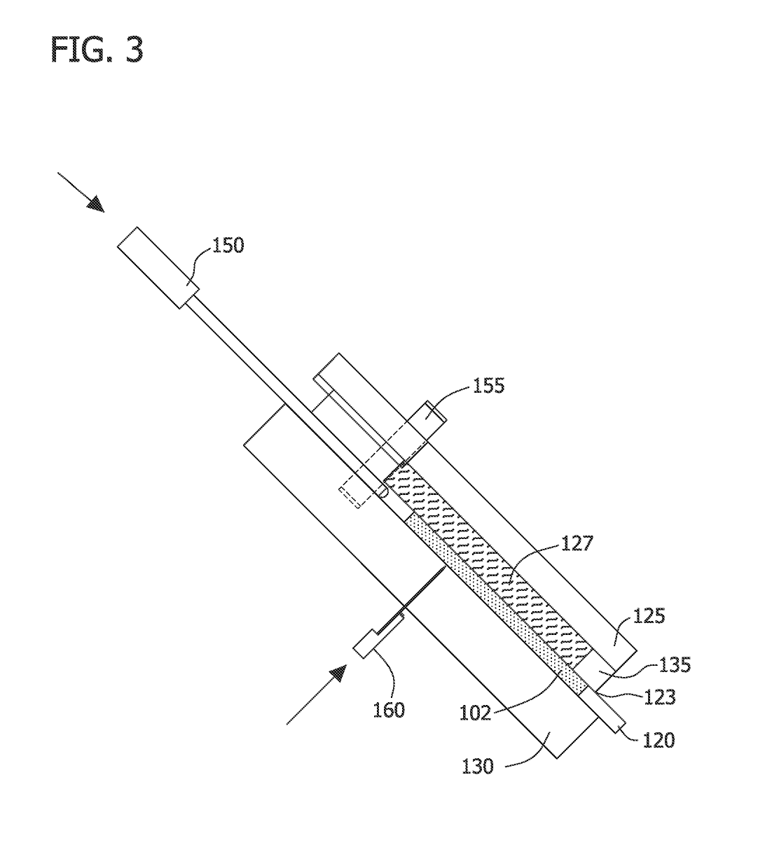

Referring now to FIG. 2, the testing assembly 115 comprises a suitable support structure 140 having a mounting fixture 165 supported thereby. A packet retention assembly, indicated generally at 118, is supported by the mounting fixture 165 and includes a pair of opposed holding panels 125, 130 (see also FIG. 3) disposed in transversely spaced relationship with each other to define a gap therebetween. In the illustrated embodiment, the packet retention assembly 118 is more suitably moveably supported on the mounting fixture 165 by a positioning device 135, such as a pneumatically operated linear translation device, to permit translating movement of the packet retention assembly 118 relative to the mounting fixture 165 for reasons which are described later herein.

The holding panels 125, 130 are suitably movable transversely relative to each other to increase or decrease the transverse spacing or gap therebetween. For example, in the embodiment illustrated in FIGS. 2 and 3, one holding panel 130 defines a fixed holding panel that remains fixed to the mounting fixture 165 while the other holding panel 125 is movable relative to the mounting fixture 165 (in addition to movement due to the positioning device 135) as well as relative to the fixed holding panel. It is understood, however, that both holding panels 125, 130 may be movable relative to the mounting fixture 165 (in addition to movement due to the positioning device 135) and remain within the scope of this disclosure. In one embodiment, movement of the movable holding panel 125 is by a controllable pneumatic actuator. However, movement of the holding panel 125 (and in some embodiments the other panel 130) may be by other suitable actuating devices, such as an electromagnetic actuator, hydraulic actuator, electric actuator and the like. Holding panels 125, 130 may be fabricated from any suitable material, such as plastic, metal, composites or the like. In a preferred embodiment, movable panel 125 includes a rubber layer 127 defining the inner surface of the panel.

In one particularly suitable embodiment, the movable holding panel 125 is movable relative to the fixed holding panel 130 (or, more broadly, the holding panels are movable relative to each other) for configuring the holding panels between an open configuration, a clamping configuration and a test configuration. As used herein, in the open configuration the movable holding panel 125 is spaced a relatively greater transverse distance from the fixed holding panel 130 to facilitate insertion of the packet 102 therebetween. As one example, the gap between the movable holding panel 125 and the fixed holding panel 130 in the open position may be approximately 10 mm, or any dimension greater than the test position. In the clamping position, the movable holding panel 125 is closely spaced from or even partially in abutting relationship with the fixed holding panel 130 to facilitate clamping of the packet 102 therebetween. As one example, the gap between the movable holding panel 125 and the fixed holding panel 130 in the clamping position may be approximately 0 mm to about 7 mm and may vary along the heights of the panels based on the varying thickness of the packet 102 therebetween. In particular, the holding panels may contact each other (i.e., a 0 mm gap) at the top edge of the packet or slightly above the top edge, and increase in spacing toward the bottom edge of the packet (e.g., around 7 mm). In the test position, the movable holding panel 125 is spaced from the fixed holding panel 130 at a distance greater than in the clamping position but lesser than in the open position to facilitate testing of the substance in the packet 102. As one example, the gap between the movable holding panel 125 and the fixed holding panel 130 in the test position is approximately 5 mm, or the thickness of the packet 102. It is understood that the gap between the holding panels 125, 130 may be other than as set forth above, such as depending on the thickness of the packet 102 being tested, without departing from the scope of this disclosure.

In another embodiment, one or more of holding panels 125 and 130 include one or more holes (not shown) that are in fluid communication with a source of vacuum. The holes are located in the holding panels 125, 130 such that when a packet 102 is posited between the holding panels, one or more of the holes is positioned adjacent the packet.

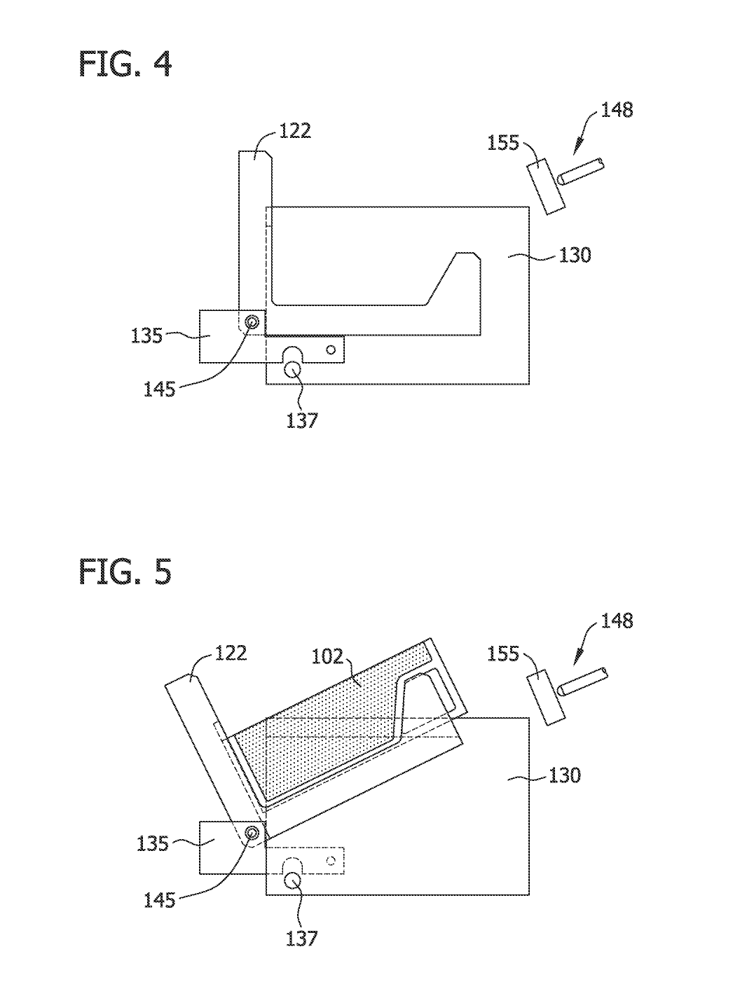

Still referring to FIG. 2, a generally L-shaped lever arm 120 is disposed at least in part between the holding panels 125, 130 for supporting the test packet 102 therebetween. More particularly, the lever arm 120 has a support arm 121 that extends axially between the holding panels 125, 130 and an actuating arm 122 that extends at least in part outward from between the holding panels to allow an operator of the testing apparatus 100 to grip the actuating arm 122. While not shown in the drawings herein, the support arm 121 in one suitable embodiment includes a groove disposed therein along all or part of its length to receive an edge of the packet to thereby facilitate proper placement and retention of the packet 102 in the testing assembly 115. In the illustrated embodiment, a suitable lever mount 123 (FIG. 3) is secured to the lever arm 120 for disposition between the holding panels 125, 130, and the lever arm 120 is pivotably mounted to the lever mount at pivot point 145 such that the lever arm 120 is pivotable relative to the holding panels (e.g., using the actuating arm 122) between a first angular position (FIG. 4) in which the support arm 121 is lowered relative to the holding panels and generally aligned axially with the holding panels, and a second angular position (FIG. 5) in which the support arm is raised relative to the holding panels to facilitate loading and unloading of the packet 102 into and from the testing assembly 115.

As best illustrated in FIG. 2, the testing assembly 115 and in particular the holding panels 125, 130 are supported by the mounting fixture 165 in an angular orientation such that the packet 102 supported by the testing assembly is oriented at an angular orientation relative to horizontal during testing of the packet. For example, in the illustrated embodiment, the holding panels 125, 130 and hence a test packet 102 supported by the holding panels and lever arm 120 during testing are at an angle relative to horizontal that maximizes the depth of the material being tested, for example about 45 degrees. It is understood, however, that the packet may be supported by the testing assembly 115 at any suitable angular orientation during testing, including horizontal or vertical, without departing from the scope of this disclosure.

With reference to FIGS. 2 and 8-16, the testing assembly 115 further comprises one or more squeezing members operable to squeeze the packet 102 prior to testing to agitate the liquid within the packet. In the illustrated embodiment the one or more squeezing members comprise a pair of mixing bars 175, 180 disposed generally within the fixed holding panel 130 and movable transversely of (e.g., inward of and outward away from) the fixed holding panel 130 within the gap between the fixed holding panel and the movable holding panel 125. More particularly, the mixing bars 175, 180 are movable between a first, retracted position (e.g., FIGS. 9 and 10) in which the mixing bars 175, 180 are disposed within the fixed holding panel 130, and a second, squeezing position (FIGS. 12 and 16) in which the mixing bars 175, 180 are in closely spaced relationship with the movable holding panel 125 within the gap between the panels to thereby squeeze the packet 102 between the mixing bars 175, 180 and the movable holding panel 125. It is understood that the mixing bars 175, 180 need not be retracted entirely within the fixed holding panel 130 in the retracted position thereof, as long as little or no squeezing of the packet is applied by the mixing bars in their retracted position. Movement of the mixing bars 175, 180 may be by any suitable actuating device, such as a pneumatic actuating device, electromagnetic actuating device, hydraulic actuating device, electric or electromechanical actuating device and the like. Although mixing bars 175, 180 are shown to be movable transversely, mixing bars 175, 180 may be configured to impart a kneading motion using a rolling, or lateral application of force to the packet.

In the illustrated embodiment, mixing bars 175 and 180 are spaced apart from each other at a distance corresponding generally to the length of the interior of the packet 102 so as to apply a squeezing force to opposite longitudinal ends of the packet. Accordingly, the mixing bar 180 is angled relative to the other mixing bar 175 to correspond generally with the configuration of the packet 102 at the end to which the mixing bar 180 applies a squeezing force to the packet. It is understood that the mixing bars 175, 180 may be oriented other than as illustrated depending on the configuration of the packet 102 to be tested. The mixing bars 175, 180 are suitably driven for movement independent of each other, and in particular to be driven in an alternating sequence in which mixing bar 175 is moved to its squeezing position while mixing bar 180 is in its retracted position (FIGS. 12 and 13) and then mixing bar 175 is moved to its retracted position which mixing bar 180 is moved to its squeezing position to impart a generally kneading action to the packet. It is understood that a single squeezing member, or more than two squeezing members, may be used and remain within the scope of this invention. It is also understood that other suitable devices may be used to squeeze or otherwise agitate the contents of the package without departing from the scope of this invention.

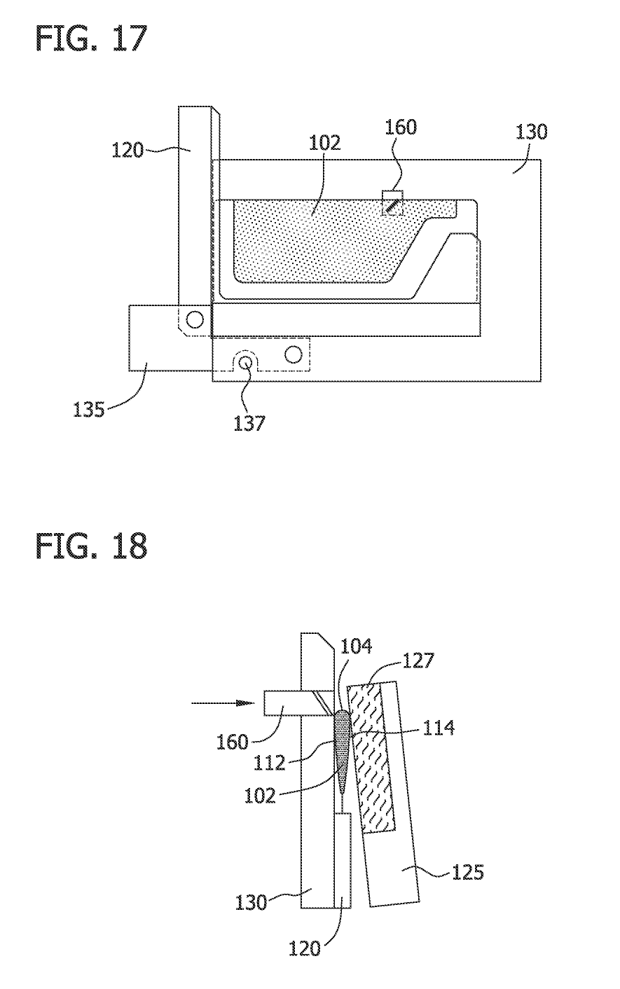

With reference now to FIGS. 17-20, the testing assembly 115 also includes a cutter 160 moveable relative to and through the fixed holding panel 130 to form a slit 185 or opening in the packet. The illustrated cutter 160 is a cutting blade translated relative to the fixed holding panel 130 by a suitable actuating device such as a pneumatic actuating device, electromagnetic actuating device, hydraulic actuating device, electric or electromechanical actuating device and the like. In other embodiments, the cutter 160 may be a knife, pin, needle, saw, laser, or other suitable cutting device operable to pierce or otherwise provide an opening in the top edge 104 of the packet. In the illustrated embodiment, the cutter 160 is configured to pierce the packet 102 a distance inward from the edge of the packet sufficient to create an opening sized to allow the probe 150 to enter the packet. In the exemplary embodiment, the cutter 160 is configured to pierce packet 102 approximately 6.5 mm inward of the top edge 104 of the packet and is angled so as to cut outwardly (i.e., upwardly in the illustrations) to the top edge 104 of the packet. The cutter 160 suitably cuts through both opposing sidewalls 112, 114 (FIG. 18) of the packet 102.

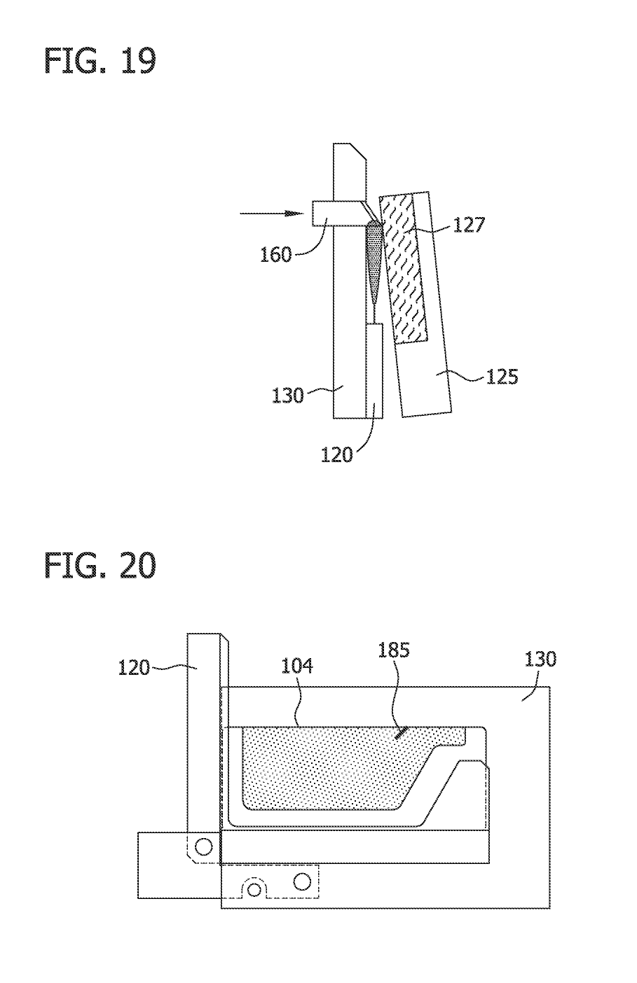

As best illustrated in FIGS. 17 and 20, the cutter 160 is oriented at an angular orientation relative to the holding panels 125, 130, and hence the packet 102 held therebetween, so that the slit cut by the cutter is at an angle relative to the packet. For example, in the illustrated embodiment the angle of the slit relative to the packet is about 45 degrees so that the slit generally forms a notch in the edge of the packet. In one embodiment, the cutter 160 is positioned at an angle with respect to the packet 102 to cut an angled notch 185 in the top edge 104. In a preferred embodiment, the cutter 160 is positioned to cut a notch 185 at a 45 degree angle with respect to the top edge 104 of the packet 102. In other embodiments, the cutter 160 may be positioned at any suitable angle of between 0 degrees and 180 degrees with respect to the top edge 104 of the packet 102 without departing from the scope of this disclosure.

As illustrated in FIGS. 1-3 and FIGS. 21 and 22, the testing assembly 115 further includes a probe assembly 148 mounted on the mounting fixture 165 by suitable mounting structure (not shown). The probe assembly 148 comprises a probe 150 operatively connected to a suitable actuating device for movement relative to the holding panels 125, 130 and hence the packet held therebetween. The probe 150 includes a mount 200 for operatively connecting the probe 150 to the actuating device, a shaft 205 extending axially from the mount and a probe tip 190 at the distal end of the shaft. In the illustrated embodiment, the probe 150 (and in particular the probe shaft 205) is oriented at an angle relative to the longitudinal axis of the packet 102 to facilitate maximum insertion of the probe shaft 205 into the packet 102 (e.g., at an angle along the interior of the packet) through notch 185. For example, the probe shaft 205 may suitably be at an angle between about 10 and about 45 degrees angle relative to the longitudinal axis of the packet 102, and more suitably about 23 degrees. It is understood, however, that the probe 150 may be at any angular orientation relative to the packet 102 without departing from the scope of this disclosure.

The probe shaft 205 according to one embodiment may be approximately 127 mm long. However, the probe 150 length may vary within the scope of this disclosure, as long as it is sufficiently long to extend into the packet 102 and be immersed in the liquid therein. The illustrated probe shaft 205 is fabricated from an epoxy material with the probe tip 190 being fabricated from glass. However, the probe shaft 205 and probe tip 190 may be fabricated from any suitable material that allows the testing apparatus to function as described herein. The probe assembly 148 also includes a probe cleaning device 155, such as a gasket, wipe, spray device or other suitable cleaning device, to apply a cleaning solution to or otherwise clean the probe 150 after the probe is withdrawn from the packet 102.

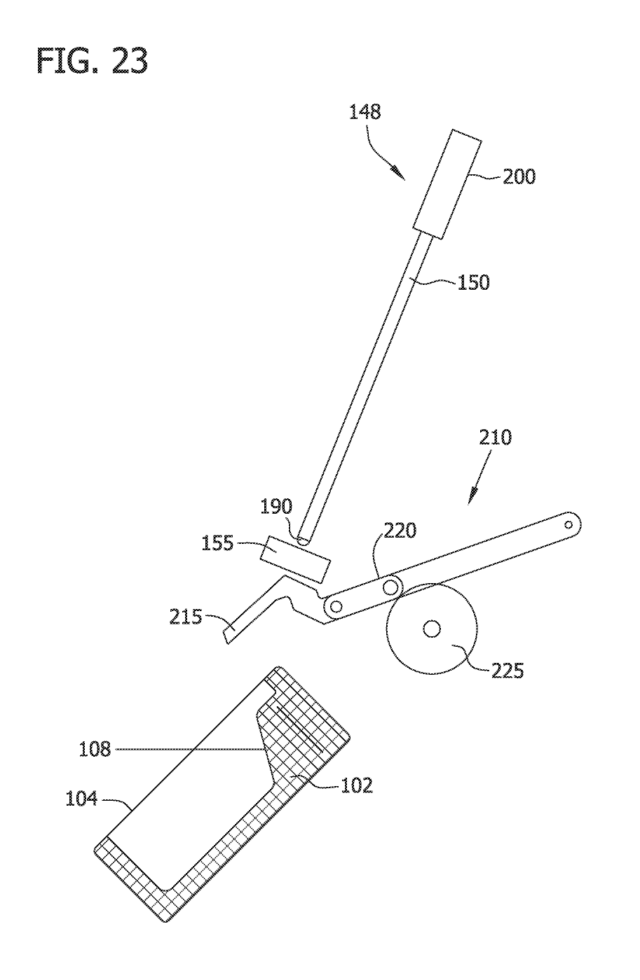

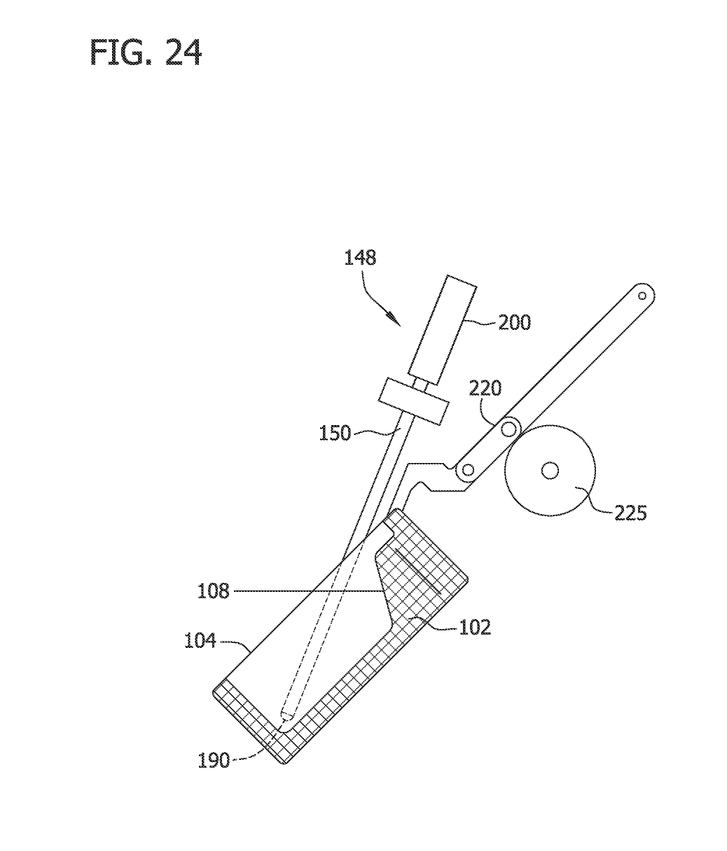

With reference now to FIGS. 23 and 24, a hold down device, generally indicated at 210, of the testing assembly 115 includes a hold down finger 215 and a linkage 220 operatively connecting the hold down finger to a pivot 225. In this manner the hold down device 210 is positionable between a raised position (FIG. 23) in which the hold down finger 215 is spaced from the packet 102 and a lowered position (FIG. 24) in which the hold down finger is disposed between the holding panels 125, 130 in contact with a portion of the packet 102 above the notch 185 and may also function to hold the packet 102 in place within the testing assembly 115 during testing—and in particular during insertion and removal of the probe 150 into and from the packet 102. When the hold down finger presses against the packet 102 above the notch 185, the notch is opened for easy insertion of the probe 150 into the packet.

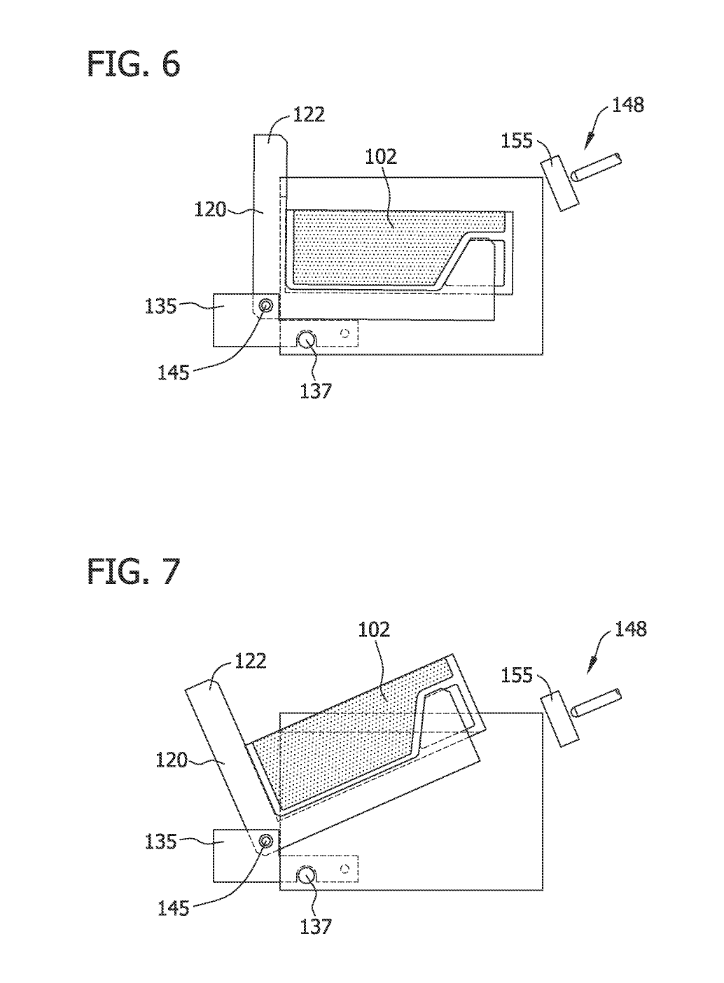

In operation according to one embodiment of a method for detecting spoilage of the contents of a flexible packet such as the packet 102, FIGS. 4-7 illustrate a loading and unloading sequence of a packet 102 into the testing assembly 115. At the initiation of a test sequence, the movable holding panel 125 is in its open configuration spaced from the fixed holding panel 130 to facilitate loading of the packet 102 into the testing assembly 115. The lever arm 120 should be in its second or rotated position, but to the extent that it is not (e.g., during storage or other inoperation of the testing apparatus 100 as shown in FIG. 4), the test operator pulls on the actuating arm 122 to pivot the lever arm 120 about hinge 145 from the first position illustrated in FIG. 4 to the second or rotated position illustrated in FIG. 5. The operator then inserts a packet 102 onto the support arm 121 of the lever arm 120 with an edge of the packet seated in the groove (not shown) formed in the support arm of the lever arm. Once the packet 102 is seated on the lever arm 120, the operator pushes the actuating arm 122 to rotate lever arm 120 about hinge 145 back to the first position of the lever arm, as illustrated in FIG. 6.

With the packet 102 now disposed between the holding panels 125, 130 and supported by the lever arm 120, the operator may then initiate an automated testing sequence. In particular, the operator may press a start button 170 (FIG. 1) or other suitable actuator on the control assembly 110. Start button 170 may, for example, be a physical button, a button on a touch screen, a computer clickable button or any other suitable actuator for starting the testing sequence.

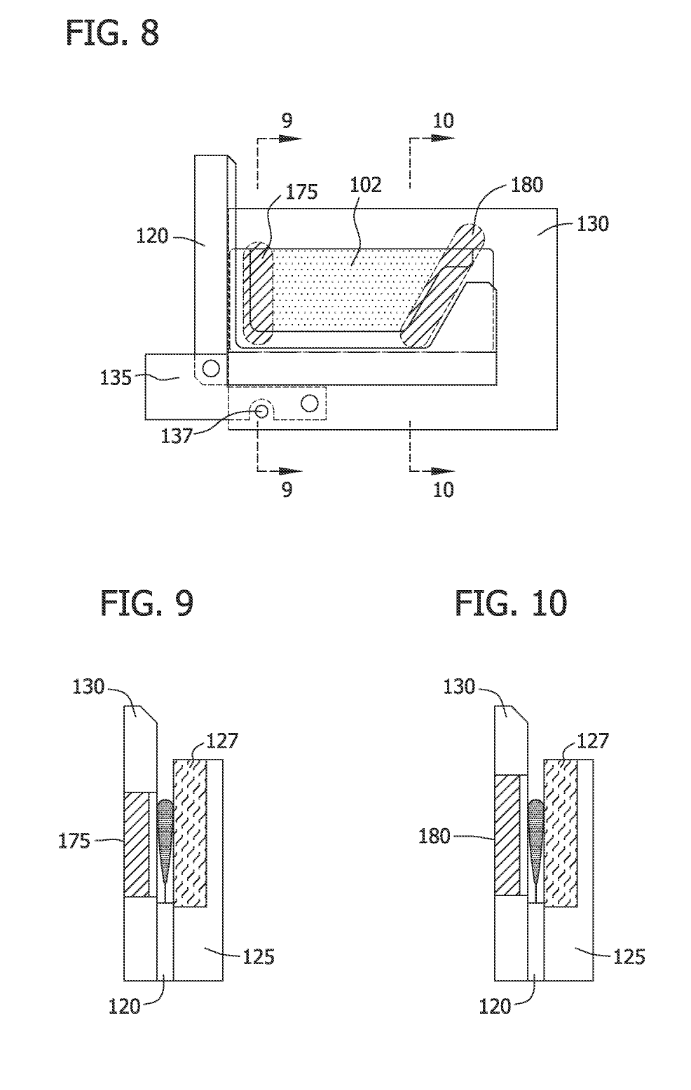

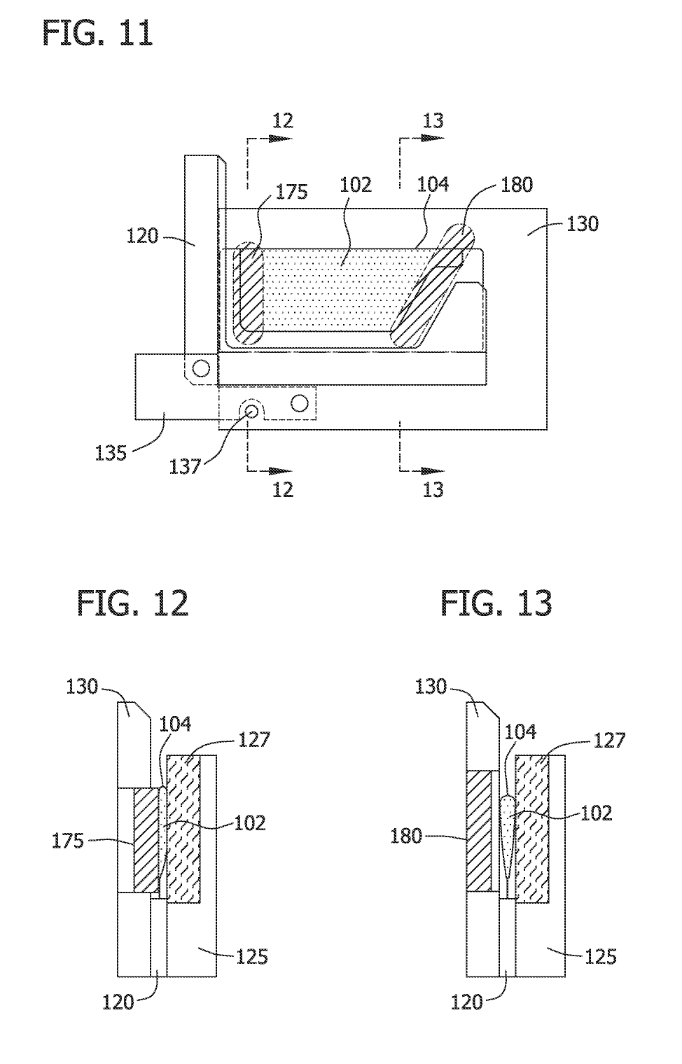

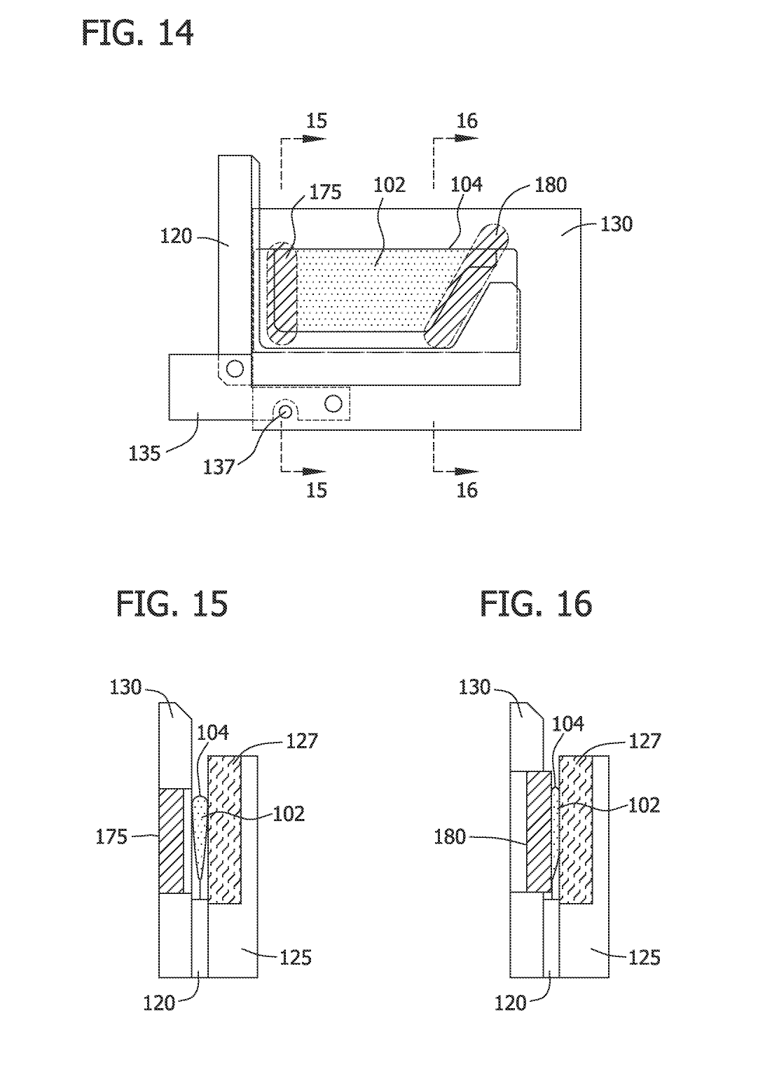

After depressing the start button 170, the control system operates the movable panel 125 to move to its clamping position. In another embodiment, the movable panel 125 may instead be actuated to move to its test position. Initially, positioning device 135 is in its lowermost position, abutting the stop 137. Subsequently, a kneading (e.g., squeezing) operation is initiated to facilitate mixing of the contents of the packet 102. With reference to FIGS. 8-16, FIG. 8 illustrates the packet 102 before initiation of the kneading operation. Mixing bars 175 and 180 are both in their retracted positions in which they do not squeeze or apply a substantial pressure to packet 102. The control system then operates to move mixing bar 175 to its squeezing position to squeeze one end of the packet 102, as illustrated in FIGS. 11-12 (the other mixing bar 180 remaining in its retracted position as illustrated in FIG. 13). After a predetermined time has elapsed, mixing bar 175 is retracted back into fixed wall 130 (FIG. 15) and the control system operates to move mixing bar 180 to its squeezing position as illustrated in FIGS. 14 and 16 to squeeze the opposite end of the packet 102. The alternating sequence of moving mixing bars 175 and 180 to their respective squeezing positions is repeated a predetermined number of times, such as 5 complete cycles, to facilitate proper mixing/homogenization of the contents of the packet 102 prior to testing. In one embodiment, the number of kneading cycles is user programmable and may be set to any number of cycles.

After the kneading operation is complete, a locating operation is initiated to locate the top edge 104 of packet 102 and position it at a predetermined location relative to the cutter 160 and the probe assembly 148 (i.e., because each packet 102 may vary in size such as due to manufacturing operations and tolerances). The positioning device 135 is operated to move at least the a lever arm 120 and packet 102 relative to the holding panels 125, 130. For example, the positioning device 135 initially has the lever arm 120 positioned at its lowest position at which the device contacts a stop 137. During the locating operation, the positioning device 135 raises the lever arm 120 and hence the packet 102 until a position sensor (not shown) detects the top edge 104 of the packet 102 as being at a predetermined height corresponding to a desired position relative to the cutter 160 and probe assembly 148. In one embodiment, the position sensor may be an optical sensor that detects when an optical beam has been interrupted by the top edge 104 of the packet 102, indicating that the top edge 104 of packet 102 is in the desired position. In other embodiments, the position sensor may be any sensor, such as an acoustic, magnetic, tactile or other suitable sensor that allows the testing assembly 115 to operate as described herein.

Once the sensor detects that the top edge 104 of the packet 102 is at the desired position, the lever arm 120 is maintained at that position, as illustrated in FIGS. 17 and 18. In one embodiment, if not already done previously, the movable panel 125 is moved to its clamping position, as illustrated for example in FIGS. 18 and 19, to provide additional stability to packet 102 when being cut by cutter 160. Subsequently, the control system causes cutter 160 to move in the direction toward and through packet 102 as illustrated in FIG. 19 to cut a slit or notch 185 (FIG. 20) in the top edge 104 of the packet 102. The cutter 160 is then retracted away from the packet 102 to its initial retracted position (i.e., the position before cutting takes place).

After the notch 185 has been cut, vacuum is applied to opposite sidewalls 112, 114 of the packet 102 via the vacuum openings (e.g., holes) (not shown) in inner surfaces of the fixed holding panel 130 and movable holding panel 125. The vacuum causes the sidewalls 112 and 114 of packet 102 to be drawn outwardly against the fixed holding panel 130 and the movable holding panel 125. With the sidewalls 112, 114 of the packet held by the vacuum to the holding panels 125, 130, the movable holding panel is then moved to its test position, i.e., spaced further from the fixed holding panel, to generally pull the sidewalls of the packet apart to thereby generally open the packet at the notch 185 so as to facilitate insertion of the probe 150 into the packet.

The hold down device 220 is actuated to pivot the hold down finger 215 to its lowered position (FIG. 24) in which the hold down finger is disposed between the holding panels 125, 130 in contact with the packet 102 to hold the packet in place.

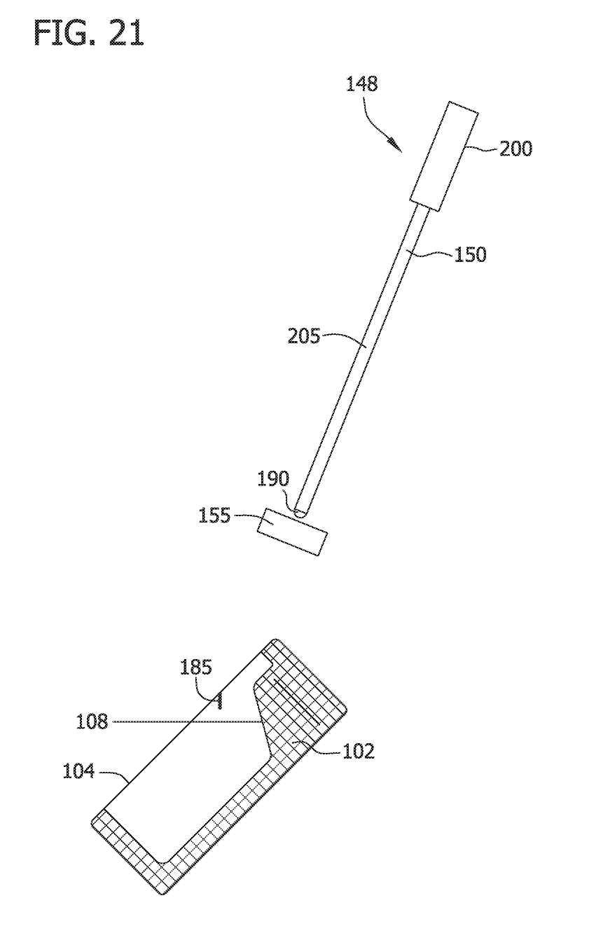

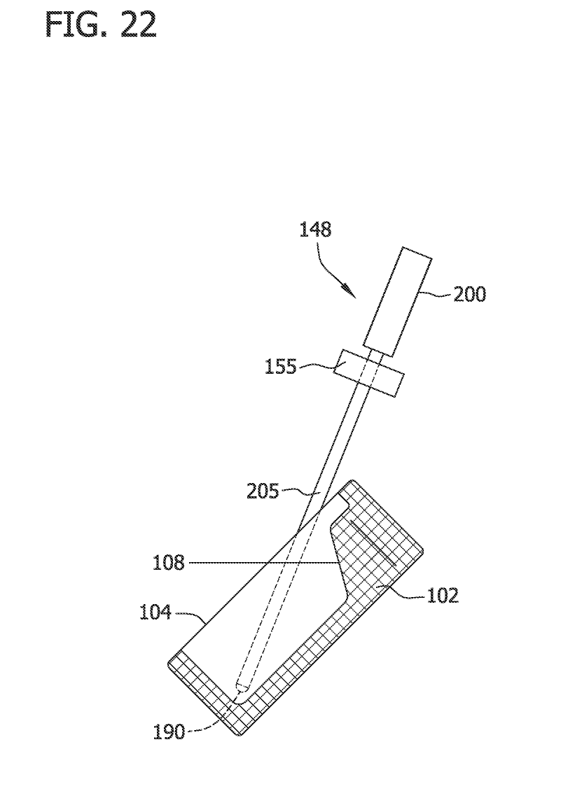

FIG. 21 illustrates the packet 102 prior to probe insertion. For ease of understanding, the various components of the testing assembly 115 other than the probe assembly 148 and packet 102 are omitted from FIGS. 21 and 22. The fluid level of the packet 102 is illustrated by fluid level line 108.

To test for spoilage of the contents of packet 102, the control system operates an actuator (not shown) to move the probe 150 toward packet 102, such that the probe tip passes through notch 185 into the interior of the packet a predetermined distance as illustrated in FIG. 22 to assure that the tip 190 is in the liquid contents of the packet. In one embodiment, the probe tip 190 is inserted into the packet 102 at least about 50 percent of the length of the packet 102. The probe tip 190 is then used to sense at least one characteristic of the contents of the packet wherein the at least one characteristic indicative of spoilage of the contents. As one example, the probe tip 190 samples a pH of the contents. However, it is understood that other characteristics of the packet contents may be sensed by the probe 150 and used to assess spoilage of the contents. The pH is compared by the control system to a predetermined characteristic range, and if the tested pH of the contents of the package are outside of the range, the control assembly provides an audible or visual alert to the operator indicating that the contents of the packet 102 are spoiled. In particularly suitable embodiments, the at least one characteristic is also recorded and saved to memory in the control system and/or transmitted to a remote computer system. In other embodiments, probe 150 may be configured for and used to assess one or more characteristics of the contents, for example, density, electrical conductivity, color, moisture content, temperature or the like.

In one embodiment, the testing assembly 115 may further include a code scanner that reads indicia or any other suitable indicator on the packet 102 that relates to a batch number, product code or other identifier associated with the contents of the packet. In one embodiment, this information is recorded along with the sampled characteristic of the packet contents.

After the characteristic reading is taken, the probe 150 is retracted from the packet such that the probe shaft 205 and tip 190 are drawn through the probe cleaning device 155. The probe cleaning device 155 applies a cleaning solution to the probe to clean the contents of the packet 102 off of the probe shaft 205 and tip 190. In a more particularly suitable embodiment where the characteristic sensed by the probe 150 is the pH of the contents, the cleaning device 155 applies a buffer solution to the probe shaft 205 and probe tip 190. The buffer solution has a pH that is outside of a pH range of the contents of the packet 102 in a normal or spoiled condition. For example, if the normal pH level of the contents of the packet is between 5 and 6, a buffer solution having a pH greater than 6 or less than 5 may be selected. Thus, the pH of the buffer solution may be compared to the sampled pH of the contents of the packet 102 to indicate whether the probe actually entered into the packet and took a reading of the contents. For example, subsequent to cleaning, the pH of the cleaned probe is sampled and compared to the pH sampled from packet 102. If the control assembly determines that the pH of the probe tip 190 after cleaning is the same as the pH of the probe tip after sampling the contents of packet 102, the control assembly 11 may provide an audible or visual alert to the operator indicating that the probe did not take a sample of the contents of the packet, for example, if the probe did not enter the packet 102 during the testing operation. In this instance, the operator may choose to take a manual test of the characteristic of the packet 102.

After the probe has been retracted, after testing the contents of packet 102, the hold down finger 215 is moved back to its initial state (FIG. 23) in which it is out of contact with the packet 102.

After the characteristic has been tested and optionally recorded, the testing procedure has been completed. To remove the packet 102, the movable holding panel 125 is moved to its open position to increase the gap between the movable holding panel 125 and the fixed holding panel 130. The operator then pulls/pushes on the actuating arm 122 of the lever arm 120, which pivots the lever arm 120 to its second or rotated position as illustrated in FIG. 7. The user may then grasp, remove and discard the sampled packet 102.

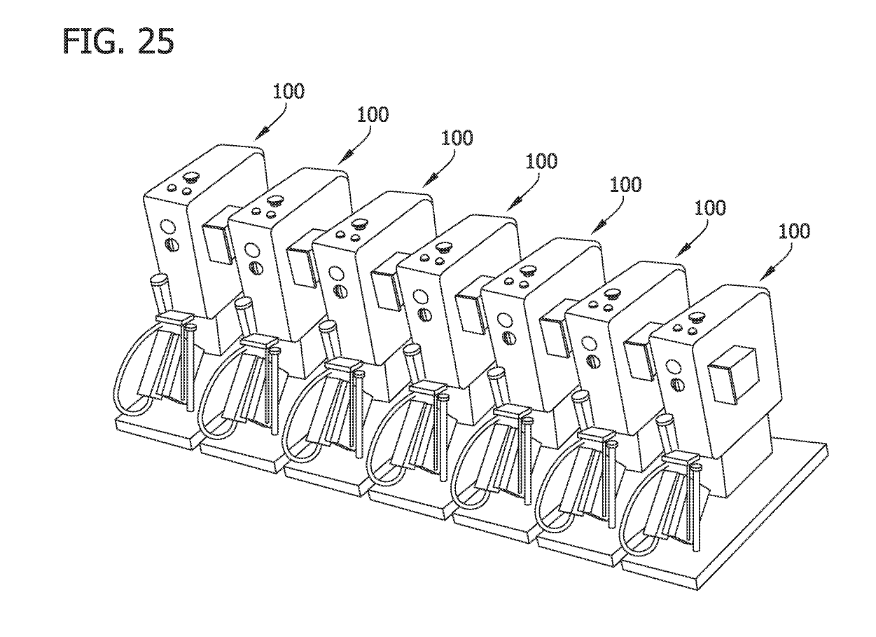

In one embodiment, as shown for example in FIG. 25, a plurality of testing apparatus 100 are positioned in close proximity to one another to allow a user to perform multiple simultaneous packet testings. The plurality of apparatus 100 may be in communication with a common computer system, either directly or by wireless communication, to receive and store data from each apparatus. In a preferred embodiment, each of the plurality of testing units are in communication with a common start button, such that multiple testings of packets may be initiated with a single button press. In an alternative embodiment, each testing apparatus 100 is operated independently.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of detecting spoilage of the contents of a flexible packet, the packet having opposed sidewalls and a longitudinal axis, the method comprising:
locating the packet between a pair of opposed holding panels with the packet oriented generally parallel to said holding panels such that one packet sidewall faces one of said holding panels and the opposite packet sidewall faces the other holding panel;
agitating the packet while retaining the packet between the opposed panels to thereby agitate the contents of the packet;
forming an opening in the packet;
inserting a probe through said opening into the interior of the packet; and
operating the probe to assess at least one characteristic of the contents of the packet, said at least one characteristic being indicative of spoilage of said contents of the packet.

2. The method of claim 1 wherein agitating the packet comprises squeezing the packet between the holding panels.

3. The method of claim 2 wherein squeezing the packet comprises applying alternating squeezing forces to the packet at least at two longitudinally spaced locations along the packet.

4. The method of claim 1 wherein inserting the probe comprises inserting the probe into the packet at an angle in the range of about 10 degrees to about 45 degrees relative to the longitudinal axis of the packet.

5. The method of claim 1 wherein the at least one characteristic comprises the pH of the contents of the packet.

6. The method of claim 1 further comprising withdrawing the probe from the packet after assessing the at least one characteristic of the contents of the packet, and cleaning the probe as the probe is withdrawn from the packet.

7. The method of claim 1 wherein locating the packet between a pair of opposed holding panels comprises moving at least one of said holding panels away from other one of said holding panels, locating the packet between the holding panels, and moving said at least one of the holding panels back towards said other one of said holding panels to retain the packet therebetween.

8. An apparatus for detecting spoilage of the contents of a flexible packet, the packet having opposed sidewalls and a longitudinal axis, the apparatus comprising:
a pair of holding panels disposed in opposed spaced relationship with each other to define a gap therebetween in which the packet is retained with one packet sidewall facing one holding panel and the other packet sidewall facing the other holding panel;
at least one squeezing member operable to squeeze at least a portion of the packet while the packet is retained between said holding panels;
a cutting member movable relative to the holding panels to cut at least one inlet in the packet; and
a probe moveable relative to the panels for insertion through said at least one inlet in the packet to position the probe within the packet, the probe being operable to assess at least one characteristic of the contents of the packet, said at least one characteristic being indicative of spoilage of the contents of the packet.

9. The apparatus of claim 8 wherein the squeezing member comprises a pair of bars disposed in spaced relationship with each other and being moveable relative to the holding panels within the gap therebetween to squeeze the packet.

10. The apparatus of claim 8 further comprising a finger member positionable relative to the holding panels between a first position in which the finger is spaced from the packet disposed between the holding panels, and a second position in which the finger is disposed in the gap between the holding panels and contacts the packet to inhibit movement of the packet upon insertion of the probe into the packet.

11. The apparatus of claim 8 wherein the holding panels are configured to retain the packet at a first angular orientation, the probe being oriented for axial movement relative to the holding panels at a second angular orientation different from the first angular orientation.

12. The apparatus of claim 8 wherein at least one of the holding panels is moveable relative to the other one of the holding panels to adjust the gap therebetween.

13. The apparatus of claim 8 further comprising a probe cleaning device.

14. A testing system for detecting a characteristic of the contents of a plurality of flexible packets, the packets each having opposed sidewalls and a longitudinal axis, the system comprising:
a plurality of testing apparatus arranged in close proximity to each other, each testing apparatus being in communication with a common computer system and capable of transmitting data to the computer system;
wherein each testing apparatus comprises:
a testing assembly configured to retain at least one packet therein during testing;
a cutting member movable relative to the holding panels to cut at least one inlet in the packet; and
a probe moveable relative to the packet for insertion through said at least one inlet in the packet to position the probe within the packet, the probe being operable to assess at least one characteristic of the contents of the packet and the operable to communicate the characteristic to the common computer system.

15. The testing system according to claim 14 wherein each testing apparatus further comprises an agitating member operable to agitate the contents of the packet prior to inserting the probe into the packet.
